(19) United States
(12) Patent Application Publication
(10) Pub. No.: US 2007/0140923 A1
(43) Pub. Date: Jun. 21, 2007

(54) Scent dispenser

(76) Inventor: Joel Pierce Wiegand, Nunica, MI (US)

Correspondence Address: CHRISTOPHER D. HARRINGTON, SUITE 350, 5300 CORPORATE GROVE DRIVE SE, GRAND RAPIDS, MI, 49512, US

(21) Appl. No.: 11/305,972

(22) Filed: Dec. 19, 2005

(51) Int. Cl. A24F 25/00; A61L 9/12

(52) U.S. Cl. 422/124; 239/53; 239/56

(57) ABSTRACT

A novel scent dispenser comprises a body ventilated between its interior and exterior, a scent pad housed within the body, a cable for suspending the body from a point with at least one swivel at the end of the cable that attaches to the body, and with a scent pad that emits a scent selected by the user.

### CROSS REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

The present invention relates to a dispenser for emitting a scent. More particularly, the present invention relates to a novel airfoil shaped dispenser for maximizing the passive emission of animal scent, perfumes, and the like.

The usage of scent dispensers of one sort or another has long been known in the prior art. Most are passive type of emitters in that they do not use any artificial means to accelerate the emission of a scent. Non-passive devices would employ electrical power to heat or rotate the scent dispenser for instance, which then means that the user has to rely upon the existence of an electrical supply. Other non-passive methods would include the use of a specialized gun to shoot a scent container into a targeted area where the container opens to expose the scent. Non-passive scent dispensing cannot be used in many applications where it is desired to have a scent release. Chief among these considerations would be hunting applications where the noise or foreign smells associated with non-passive scent transmission would be a drawback and could actually inhibit game from approaching the area where the scent is to be disbursed.

Passive scent dispensers have been used in hunting applications and include such varied devices as inverted bottles that allow for a time-released drip of a scent material. Thus when the hunter is walking, he/she is leaving a scented trail which mimics the way a natural scent would be laid down by the particular game animal that the hunter is seeking. Another approach is the use on a horizontally oriented container that is cigar shaped in cross section and which is ventilated to allow wind currents to penetrate and lift scent from a reservoir. Another passive approach that is fairly common is the use of a flat scent “pad” that may be die-cut into a shape like a tree and then hung from a location like a mirror in a motor vehicle. The scent commences to volatize and be emitted once the package containing the pad is opened and continues until the scent is virtually exhausted from the pad. These and many other devices have been known in the prior art and still the search for an efficient and versatile method for scent dispensing continues.

One of the problems associated with the prior art is the need to augment the natural means for scent transmission. Two primary factors, wind and temperature, will dictate the speed with which scent chemistry is volatized or vaporized from a particular source. Increasing temperature serves to accelerate the vaporization process by increasing the vapor pressure of the scent chemistry. Wind currents serve to lower the effective atmospheric pressure in the area of the scent chemistry, thereby stimulating more emission from the scent chemistry. In most instances, especially with respect to hunting applications, the adjustment of atmospheric temperature is not a viable consideration. However, in some prior art applications, scent dispensers are themselves heated in order to increase emissions.

With respect to the effect of atmospheric pressure, wind currents may be created artificially through the use of fans or motors that drive the scent dispenser in a rotational action. Natural leveraging of wind currents occurs when a scent dispenser is hung or mounted in an open manner and allowed to react to wind currents. Many of the prior art devices are simply mounted in a fixed position and do allow the scent chemistry to be exposed in some fashion to the wind currents. Some are hung and rely on oblique contact with wind currents, not only to capture air flows and direct them in the vicinity of the scent chemistry, but also to use the contact with the wind currents to generate actions such as spinning or turning which further enhances the efficiency of scent emissions. Typically such devices use structure to leverage the wind currents, sometimes using purposefully unbalanced elements in order to maximize the effects of oblique air contact, or in other cases, the wind currents may be captured and then used to effectuate an action.

In either case, the reaction of such structure to wind currents is crudely inefficient in utilizing wind currents and only generates action from a small volume of the air flow, percentage-wise, that would otherwise be available to the dispensing device.

The long standing problem that persists is the maximization of scent emissions using wind currents that are not just oblique or that are captured or entrained, but to find a means where the scent dispenser can utilize oblique and laminar wind currents to good advantage and to convert the energy in the wind currents to action that enhances scent transmission.

The benefits of the present invention therefore address the problems that still persist and in so doing, provide a novel and highly convenient scent dispenser that can be used in numerous applications.

### SUMMARY OF THE INVENTION

A novel scent dispenser comprises a body that presents a vertically oriented airfoil shape to a prevailing wind current, where the body is suspended from a point by attachment to a swivel thereby allowing the body to rotate about its central longitudinal axis. Interior to the body is a scent pad which communicates with the air exterior to the body by a plurality of vents. The scent dispenser of the present invention is installable in a position where it may be impacted by wind currents both oblique and laminar, and the airfoil shape of the body interacts with both laminar and oblique wind currents to generate the aforementioned rotation.

In one embodiment of the present invention the scent pad is replaceable allowing the user to customize the scent for each particular application, In another embodiment the scent dispenser is loaded with a fragrant scent pad for the introduction of pleasant odors into a confined area such as a motor vehicle or a boat cabin. In yet another embodiment, the dispenser of the present invention is supplied with a battery powered motor drive for rotatably driving the dispenser when suspended from a point.

### BRIEF DESCRIPTION OF THE DRAWINGS

FIG. 1 is an isometric view of the scent dispenser of the present invention as suspended from a tree limb.

FIG. 2 is an isometric view of the scent dispenser of FIG. 1, with the body of the scent dispenser opened.

FIG. 3 is an isometric view of the scent dispenser of FIG. 2, showing a scent pad in alignment for installation into the scent dispenser.

FIG. 4 is a bottom end view of the scent dispenser of FIG. 1.

FIG. 5 is a top end view of the scent dispenser of FIG. 1.

FIG. 6 is a partial side view of the top end of the scent dispenser of FIG. 1.

FIG. 7 is an isometric view of a scent pad of the present invention shown in a sealed package.

FIG. 8 is an isometric view of an alternate embodiment of the scent dispenser utilizing a battery powered motor drive and as suspended from a tree limb.

FIG. 9 is a representation of the air foil shape of the dispenser of the present invention with the laminar wind currents indicated by arrows.

FIG. 10 is a representation of the air foil shape of the dispenser of the present invention with the oblique wind currents indicated by arrows and the dispenser movement generated indicated by arrow “A” and arrow “B.”

FIG. 11 is an isometric view of the dispenser of the present invention in use in a motor vehicle and as suspended from a rear view mirror.

FIG. 12 is a partial side view of the dispenser of the present invention in use in the cabin of a motor boat.

### DETAILED DESCRIPTION OF THE PREFERRED EMBODIMENTS

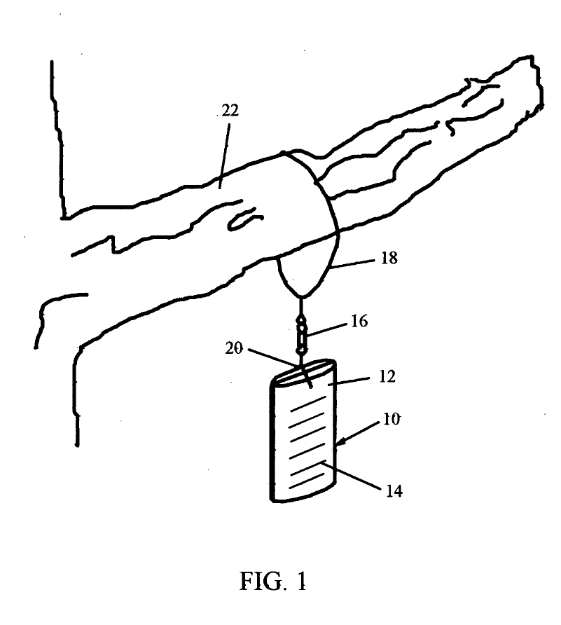

A new scent dispenser in accordance with the present invention is shown in the drawings and is discussed in detail herein. Specifically, as may be seen in FIG. 1 a scent dispenser 10 includes a dispenser body 12, a plurality of vents 14, a swivel connection 16, a hanging loop 18 and a dispenser loop 20. The scent dispenser 10 in FIG. 1 is suspended from a tree limb 22 by means of the hanging loop 18, which is connected to the swivel 16, which is then connected to the dispenser loop 22 which then is connected to the scent dispenser 10.

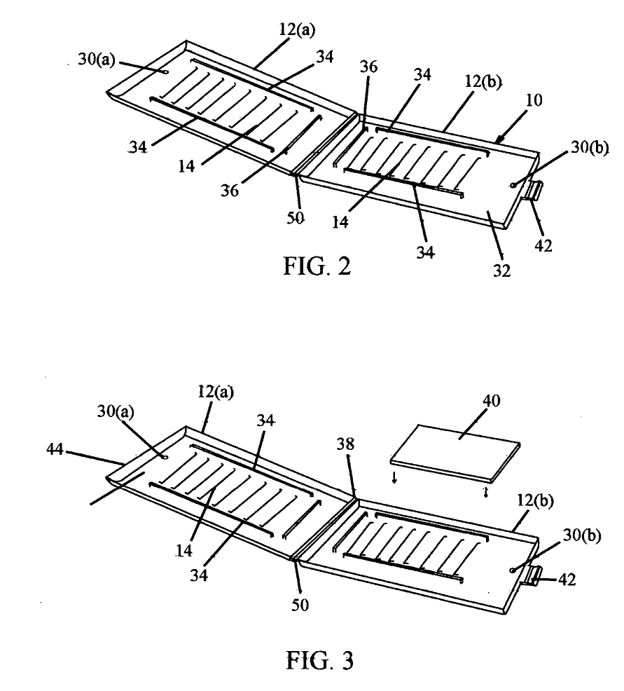

In FIGS. 2 and 3, the scent dispenser 10 with body halves 12(a) and 12(b) is revealed in more detail with the through holes 30(a) and 30(b), the dispenser interior 32, the scent pad holder side(s) 34, scent holder bottom 36, the dispenser bottom 38, the scent pad 40, the locking clasp 42, the dispenser top 44 and the living hinge 50. The scent pad 40 is positioned above the dispenser interior 32 and fits into the space defined by the scent holder sides 34 and the scent holder bottom 36.

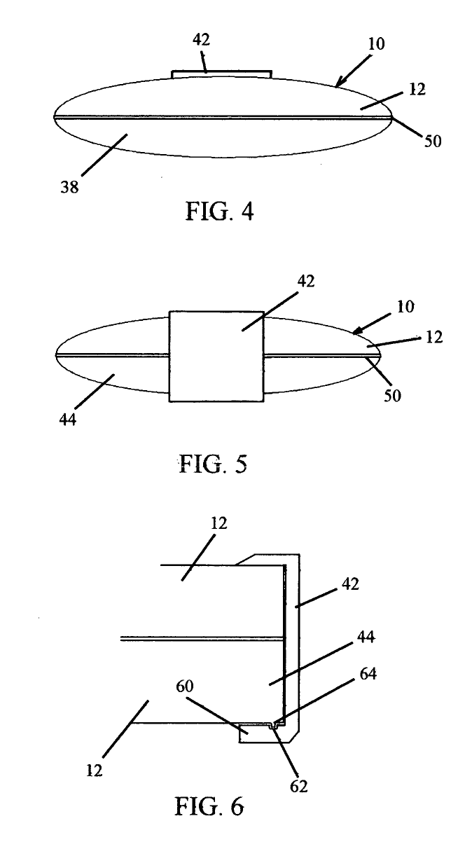

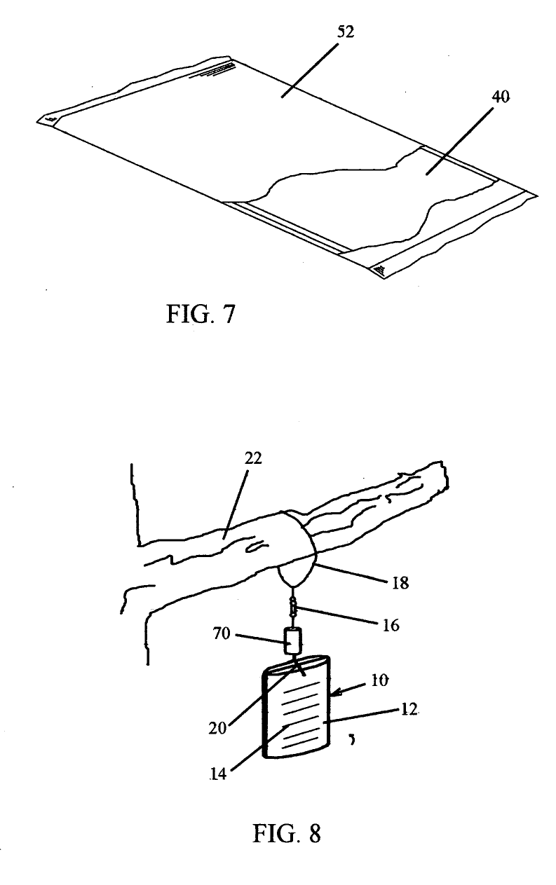

In FIGS. 4 and 5, the scent dispenser 10 is viewed from the top and bottom with the features noted above. In FTG. 6, the top portion of the scent dispenser 10 is shown in closer detail with the clasp end 60, the clasp recess 62 and the locking lip 64. FIG. 7 discloses a scent pad 40 that is packaged in a wrapper 52. As will be noted below, the scent pads 40 are kept fresher if they are individually wrapped,

In use, the scent dispenser 10 is fabricated to be a compact device, typically about “pocket-sized” and can easily be stored in a coat, a storage duffle, a hunting vest or in similar places. The scent pad 40 is an absorbent pad that may be made out of any number of materials including cotton, cotton blends, and synthetic materials all of which share the property of being able to retain scent chemistry in a releaseable fashion, The scent pads 40 are thin enough to allow some aeration when they are exposed to a wind current, thereby enhancing the release of the desired scent. They are preferentially packaged individually to preserve their scent and the use of Mylar type film for the package material is also preferred since this provides a hermetically sealed means for preserving the scent pads 40.

To install a scent pad 40 in the scent dispenser 10, the user can open the scent dispenser 10 by undoing the clasp 42 which then separates the body 12 into its halves, 12(a) and 12(b) as is best shown in FIGS. 2 and 3. The body halves are linked together by the living hinge 50 which is integrally formed with the scent dispenser 10. The area where the scent pad 40 is to be installed is bounded by the respective scent holder sides 34 and the scent holder bottom 36. As can be seen in FIG. 3 in particular, the scent pad 40 is merely laid into the holding area and then the opposing half of the scent dispenser body 12(a) can be closed thereby retaining the scent pad 40 in place. The dispenser 10 can be “locked” into the closed position by extending the clasp 42 over the dispenser top 44 and fitting the clasp recess 62 over the locking lip 64 found near the top 44 of the dispenser 10. When closed, the through holes 30 line up and allow the dispenser loop 20 to be inserted. The dispenser loop 20 is typically connected to a swivel assembly 16 which allows the scent dispenser 10 to rotate about its longitudinal axis. The use of one swivel assembly 16 certainly does allow the scent dispenser 10 to react to wind currents thereby allowing it to turn rotatably, however it has been observed that the usage of more than one swivel 16 is preferred since the frictional threshold for rotation appears to be lessened. This makes sense since the swivels 16 are not precision crafted and they may have points as they rotate where individual swivels 16 drag and can impair the rotation of the scent dispenser 10. By adding two or more swivels 16 the scent dispenser 10 will move more freely. The dispenser loop 20 is typically a length of line or cable, which may actually be coupled to form a loop or in the alternate, it may be left unlooped so as to allow the user to tie it to a point.

The scent dispenser 10 may be used as lure when hunting game. The scent pad 40 is infiltrated with a scent that attracts the subject game animal (such as a deer or elk for instance) and then the dispenser 10 is suspended from a tree limb 22 by using the hanging loop 18. The hanging loop 18 may have a clasp (not shown) of its own that would allow it to be easily installed and then removed from the tree limb 22. By suspending it in a position that allows it to be impacted by wind currents, the scent dispenser 10 will react. Any impacts of the novel airfoil design (as will be explained further below) will likely generate rotational movement. The action of the wind current itself, coupled with the multiplier effect that rotation imparts, allows a higher degree of emission of the scent from the scent pad 40 which is bathed in the wind currents and in the air imparted from the rotation by way of the vents 14.

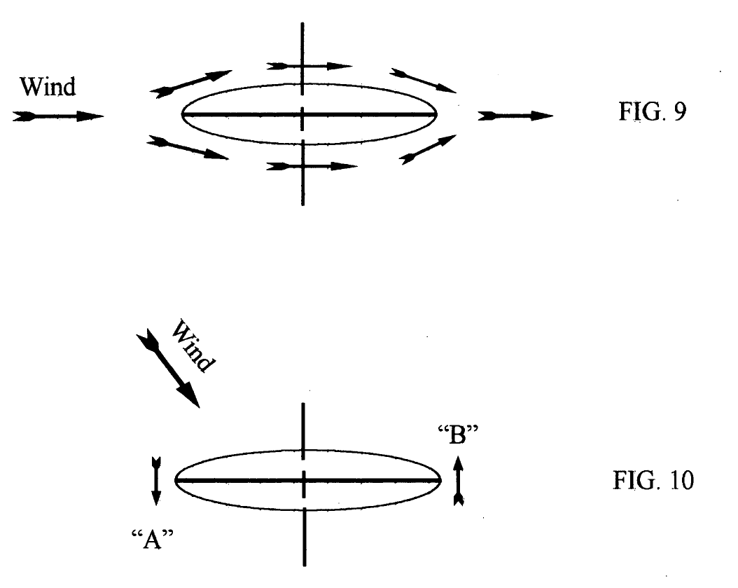

Obviously the scent dispenser 10 can be suspended from any number of different points other than a tree limb 22. The disclosure is meant to illustrate the potential and suspending the scent dispenser from tent posts, or fence wire, or any number of possible suspension points is acceptable and consistent with the objectives of the invention. While the use of the scent dispenser 10 is primarily directed towards the attraction (or repulsion) of game animals, it can also be used in confined areas in the house, in a car or elsewhere. The advantage of the present invention is in the ability to leverage wind currents more efficiently than designs known in the prior art. Reference is made to FIGS. 9 and 10, where the wind currents are diagrammatically represented. In FIG. 9, the air foil shape of the present invention creates dual slip streams of air currents when the wind direction is directly into the leading edge of the air foil. The slipstreams create pressure gradients from one side of the air foil to the other and the slightest difference in the pressure gradient will cause the scent dispenser to rotate about the longitudinal axis, which directly towards the viewer of FIG. 9. Without manual intervention, it is impossible for the airfoil to stay in a neutral position when confronting the wind current as shown, unlike other designs which have blunt or totally rounded bodies, Thus the scent dispenser 10 of the present invention is more active in response to the orientation to the wind currents as shown in FIG. 9.

Turning now to FIG. 10, the wind current acts on the air foil shape differently when the direction is oblique to the leading edge of the air foil. The scent dispenser 10 is impacted by the wind current in an imbalanced manner which causes the scent dispenser to rotate in reaction to this force. As shown in the drawing, the result is rotation in the direction indicated by arrows “A” and “B.” In some ways this action is more typical of that seen with some of the prior art device where no air foil shape is used. The advantage though is that the present invention is much more reactive to wind currents no matter which direction they impact the body 12 of the scent dispenser 10.

The alternate version of the present invention shown in FIG. 8 is an enhancement for those occasions when there is virtually no wind current whatsoever or where a temporary augmented release of scent is necessary. The scent dispenser 10 in this case employs motor drive 70 which includes a self-contained battery source. The motor drive 70 can be fixed to rotate the scent dispenser 10 at a fixed rate. Preferentially, the motor drive 70 is noise free so as to avoid any chance of spooking the game animals that are desirably being attracted.

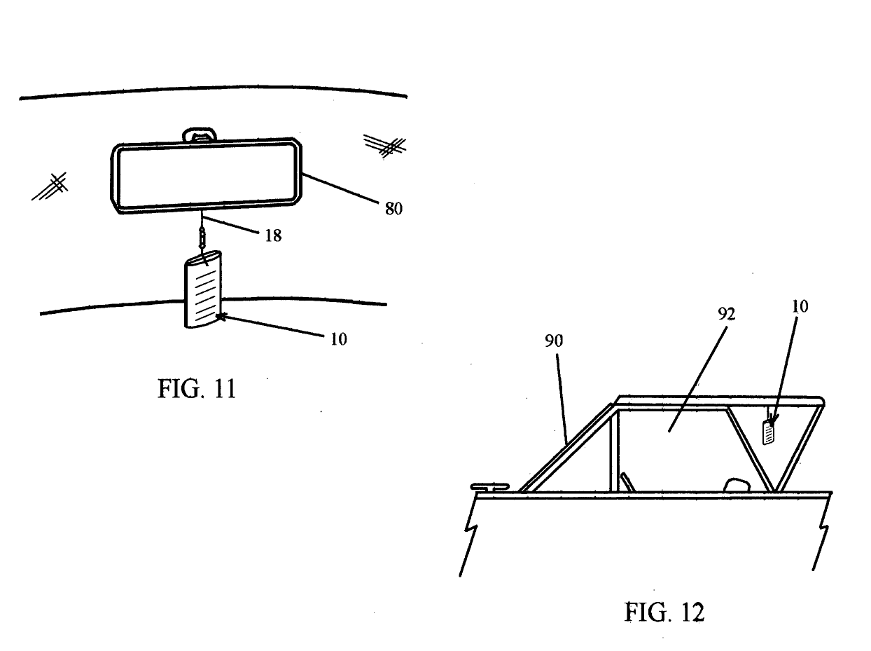

Lastly, turning to FIGS. 11 and 12, two different applications for the use of the scent dispenser 10 are shown. In FIG. 11, the scent dispenser 10 is shown as being suspended from a rear view mirror 80 in a motor vehicle. The attributes of the scent dispenser 10 discussed above still apply since wind and air currents in a typical motor vehicle are quite active. The scent pad 40 will not fill infiltrated with the scent of a game animal, obviously, and instead a fresh scent intended to impart a pleasant aroma to the interior of the motor vehicle is used instead.

Similarly, in FIG. 12 the scent dispenser 10 is suspended from a point within the cockpit 92 of a boat 90. The purposes and functions are essentially identical to those described for the use of the present invention in a motor vehicle.

Variations on the present invention can be easily realized, such as the placement of the through hole 30 on a projection (not shown) extending from the clasp 42. It is possible that only one loop, the hanging loop 18 can be used if the swivel 16 is attached directly to the body 12. It is understood that reference to the swivel 16 can mean one or more actual swivels. The present invention is shown in a form that is believed to be very compatible with plastic injection molding and can be done in great volume. The composition of the scent dispenser 10 is typically a plastic resin which can be selected from plastic resins that would be compatible for this invention by one skilled in the art.

Other variations and departures from the teachings herein can be contemplated without departing from the spirit or scope of the invention.

I claim:

1. A scent dispenser for suspension from a point, the scent dispenser emitting a scent there from, the scent dispenser of the present invention comprising;
A body with an interior and an exterior and a top and a bottom, and where said body is vented between the interior and exterior;
A scent pad containing a scent capable of being emitted from the scent pad;
A cable for suspending the body from the point with at least one swivel on one end for connecting said cable to the top of said body; and,
Where the body has a longitudinal axis running from the top to the bottom and where transverse to said longitudinal axis the body has an air foil shape, and
where said scent pad is housed in the interior of said body.

2. The scent dispenser of claim number 1, where the scent dispenser includes at least two swivels.

3. The scent dispenser of claim number 1, where the scent dispenser is comprised of a singe-piece plastic construction.

4. The scent dispenser of claim number 1, where the scent dispenser further includes a motor drive for connection between said swivel and said bottom and where the motor drive rotates the body about the longitudinal axis.

5. The scent dispenser of claim number 4, where the motor drive includes a battery power supply.

6. A scent dispenser for suspension from a point, the scent dispenser emitting a scent there from, the scent dispenser of the present invention comprising;
A body with an interior and an exterior and a top and a bottom, and where said body is vented between the interior and exterior, and where said body is comprised of foldable halves joined by a living hinge;
A scent pad containing a scent capable of being emitted from the scent pad;
At least one cable for suspending the body from the point with at least one swivel on one end of the cable for connecting the cable to the top of said body; and,
Where the body has a longitudinal axis running from the top to the bottom and where transverse to said longitudinal axis the body has an air foil shape, and
where said scent pad is housed in the interior of said body.

7. The scent dispenser of claim number 6, where the scent dispenser includes at least two swivels.

8. The scent dispenser of claim number 6, where the scent dispenser is comprised of a singe-piece plastic construction.

9. The scent dispenser of claim number 6, where the scent dispenser further includes a motor drive for connection between said swivel and said bottom and where the motor drive rotates the body about the longitudinal axis.

10. The scent dispenser of claim number 9, where the motor drive includes a battery power supply.

11. A scent dispenser for suspension from a point, the scent dispenser emitting a scent there from, the scent dispenser of the present invention comprising;
A body with an interior and an exterior and a top and a bottom, and where said body is vented between the interior and exterior, and where said body is comprised of foldable halves joined by a living hinge;
A scent pad containing a scent capable of being emitted from the scent pad;
At least one cable for suspending the body from the point with at least one swivel on one end of the cable for connecting said cable to the top of said body;
A motor drive for connection between said swivel and said bottom and where the motor drive rotates the body about the longitudinal axis; and,
Where the body has a longitudinal axis running from the top to the bottom and where transverse to said longitudinal axis the body has an air foil shape, and
where said scent pad is housed in the interior of said body.

12. The scent dispenser of claim number 11, where the motor drive includes a battery power supply.

13. The scent dispenser of claim number 12, where the motor drive includes a battery power supply.

14. The scent dispenser of claim number 11, where the scent dispenser includes at least two swivels.

15. The scent dispenser of claim number 11, where the scent dispenser is comprised of a singe-piece plastic construction.
